(19) United States
(12) Patent Application Publication
(10) Pub. No.: US 2010/0087839 A1
(43) Pub. Date: Apr. 8, 2010

(54) MESH COMPRISING ECM

(76) Inventors: Lene Feldskov Nielsen, Copenhagen K (DK); Jens Truelsen, Helsingoer (DK); Hanne Everland, Bagsvaerd (DK)

Correspondence Address: JACOBSON HOLMAN PLLC, 400 SEVENTH STREET N.W., SUITE 600, WASHINGTON, DC, 20004, US

(21) Appl. No.: 12/449,968

(22) PCT Filed: Mar. 7, 2008
(86) PCT No.: PCT/EP2008/052782

§ 371 Date: Sep. 4, 2009

(30) Foreign Application Priority Data
Mar. 7, 2007 (DK) PA 2007 00355

(51) Int. Cl. A61B 17/00

(52) U.S. Cl. 606/151

(57) ABSTRACT

The present application discloses that incorporation of dermatan sulfate and/or HA in composite scaffolds of certain polymers gives rise to a chondrogenic effect on chondrocytes resulting in formation of cartilage that resembles the natural ECM. This effect with dermatan sulfate as the primary additive has not previously been seen. The composites are formed by incorporation of dermatan sulfate finely dispersed particles optionally nanoparticles or as molecular dissolutions in a polymer matrix with no bonding between the DS and the matrix, providing the DS to the chondrocytes in an accessible non-crosslinked form.

### BACKGROUND

Abdominal wall defects can result from trauma, tumor resection or complications of previous abdominal surgery such as hernias and mesh infections. The abdominal wall comprises several muscles and facial structures that allow it to function as the protector of intra-abdominal organs and to flex and extend the trunk and support the back.

A hernia is a protrusion of a tissue, structure, or part of an organ through the muscular tissue or the membrane by which it is normally contained. Most hernias develop in the abdomen by a weakness in the abdominal wall resulting in a defect through which adipose tissue or organs covered with peritoneum protrude.

When a stoma is constructed a weakness in the abdominal wall is induced and an increased risk of a parastomal hernia are produced. This happens in 30% of all stomas with an increased incidence in colostomies compared to ileostomies and urostomies (Efron 2003).

Pushing back or reducing the herniated tissue can surgically repair most abdominal hernias. Modern reinforcement techniques involve synthetic materials (a mesh prosthesis) that avoid over-stretching of already weakened tissue spreading the mechanical load over a larger area. The mesh is placed over the defect and sometimes kept in place by staples.

Several prosthetic grafts are used for abdominal fascial repair like nonabsorbable meshes made of polypropylene (Prolene, Ethicon Inc.), polyethylene (Dacron), acrylic cloth (Orlon), polyvinyl sponge (Ivalon), polytetrafluoroethylene (PTFE, teflon mesh and cloth), expanded PTFE (Gore-tex), polyester (Mersilene) and polyvinyl cloth (Vinyon-N). Absorbable meshes include polyglactin (Vicryl, Dexon) and polyglycolic acid (Dexon). The polypropylene mesh is the most common synthetic prosthetic mesh used for abdominal repair.

Weakening of the tissue in a woman pelvic region resulting in a condition where organs fall down or slip out of place. Prolaps in the humans are either vaginal- or rectal prolaps. In vaginal prolaps a portion of the vaginate canal protrude from the opening of the vagina because either the bladder, small intestine, rectum, urethra, uterus or the roof of the vagina are protruding into vagina. In rectal prolaps the walls of the rectum protrudes through the anus and hence becomes visible outside the body.

In stress incontinence the pelvic floor muscle weakens due to physical changes resulting from pregnancy, childbirth and menopauses. The weakness results in a downward movement of the urethra when coughing, laughing, sneezing, exercising or when other movement increases the intra-abdominal pressure increasing the pressure on the bladder.

A common surgery for stress incontinence involves pulling the bladder up to a more normal position by raising the bladder and secure it with a string attached to muscles, ligaments or bone. Another possibility is to secure the bladder with a wide sling. This holds up the bladder but also compresses the bottom of the bladder and the top of the urethra, further preventing leakage.

Meshes for implants are known which adhere to cell on one site but not on the other side. This is done by lamination of the mesh with a teflon film, or by coating with a bio-absorbent material as collagen or gelatine.

Use of ECM products or meshes coated with ECM are known. These products are either in the form of a sheet or as a mesh coated by ECM components. Examples of ECM sheets consisting of lyophilized porcine ECM sheets are Surgisis Hernia Matrix, Surgisis Hernia Repair Graft and Stratasis Urethral Sling all from Cook. Examples of coating of mesh are Parietex composite mesh (polyester mesh with collagen coating) and Sepramesh (Genzyme) (polypropylene mesh with hydrogel coating consisting of sodium hyaluronate (HA), carboxymethylcellulose (CMC) and polyethylene glycol (PEG)).

### SUMMARY

Herein is disclosed the utility of discontinuous regions of Extraceullar Matrix Proteins (ECM) in promoting cell growth. When the described scaffolds are applied on top of inert materials, cell in-growth is promoted and the attachment of the mesh is properly secured.

### DETAILED DISCLOSURE

One aspect of the present invention relates to a mesh comprising a biocompatible inert material at least partly covered with a continuous material comprising discontinuous regions of ECM.

In one embodiment, the mesh is a knitted structure, preferably knitted fibers. In another embodiment, the mesh is a non-woven. In yet another embodiment the mesh is a thin porous film.

By adding discontinuous regions of ECM the coating of a mesh material it is possible to combine the range of physical properties (e.g. strength, softness, flexibility, durability) the mesh can offer with the reconstructive properties of the ECM. In addition, the price of such coating will be lower than other coatings both because the powder is a waste-product from the production of acellular ECM sheets and because the optimal amount of discrete ECM material for each application can be determined and equally distributed in the coating, hence avoiding unnecessary high concentrations of ECM. In addition to the effect of the ECM, the porous structure of the base material provides the cells with a structure for in-growth. In one embodiment a discontinuous region of ECM is obtained by adding discrete ECM material, such as particles, flakes, fibres or powder.

Meshes for implants are well described and known to the skilled person. Such meshes are typically of a biocompatible, inert material. By biocompatible is mend the ability of a material to perform with an appropriate host response in a specific application by not producing a toxic, injurious or immunological inappropriate response in living tissue. By inert is mend that it is not degrade by the surrounding bio-fluids and enzymes. In one aspect the biocompatible inert material is selected from the group consisting of PP, PE, polymers obtained by metallocene catalyzation, silicone, Teflon (fluoro carbons) and polyurethanes. A particular preferred biocompatible, inert material is propylene with an established record for such use.

The inert material may be plasma-treated in order to increase the roughness and/or obtain a functionalization on the surface and hence increase the adhesion of the cells and/or the biodegradable material.

Typically, a mesh is flat, about 0.5-1.5 mm thick. Depending on the use, it can be rounded or elongate. Independent of shape it will have two sides. If the mesh is elongated (e.g. for use as slings) it will have two ends and a middle section between the two end.

The biocompatible, inert material (often referred to as the mesh), forming the structures for slings, hernia or POP repair, can be manufactured by a broad range of different techniques. These types of structures includes: Knitted fabrics, Woven fabrics, Nonwoven fabrics (including Felted, Spun-bonded, Air-laid+calendared), Casted/blown films, and Injection moulded grids.

One aspect of the invention relates to a biocompatible inert material with ECM particles on the surface. Such material will cause cell attraction the surface. Both the cavities surrounding the ECM particles and those seen after consummation of the ECM particles will serve as anchoring points to the inert material. Thus, a method of anchoring inert materials is described.

In one aspect, the mesh has a side with reduced cellular in-growth and a side with enhanced in-growth. Combining inert materials with cell attractive materials such as ECM can do this.

The combination of cell-attractive ECM with inert materials can be obtained in different ways:
Coating an inert mesh, such as a polypropylene mesh, on one of the major sides with a biodegradable synthetic/natural polymer containing ECM particles.
Felting a mainly fibrous material having mainly inert stable fibres on one side and fibres containing ECM material on the other side.
Spun-bonding a first layer and a second layer in a sandwich structure. The first layer contains ECM material and the second don't
Film casting in two steps: a first biodegradable layer containing ECM and a second layer of an inert material.
Partial coating of an injection moulded grid
A composite material of a foam containing ECM powder and either a knitted mesh, a nonwoven fabric or a film.
Co-extrusion or coating of a inert material containing ECM particles onto another or a similar inert polymer resulting in a bicomponent monofilament of inert polymers having discreet ECM particles at the surface. This monofilament may be used for e.g. weaving or knitting. The monofilament could also be cut into stable fibers and used in nonwoven processing.
The ECM particles could also be mixed with the inert biocompatible polymer during the fiber processing giving a discrete distribution of particles, however, this would result in expensive ECM particles that are not available for the cells since only the ECM particles at the surface would be accessible for the cells. Also, the ECM particles distributed throughout the monofilament causes a weakening of the fiber.

In one aspect of the invention, the inert material is coated on one side with the continuous material comprising discontinuous regions of ECM. This is particularly advantageous for use as hernia implant. Here, the mesh should adhere only to the abdominal wall (on one side of the mesh) without adhering to the intestines.

A related aspect of the invention relates to the use of a mesh comprising a biocompatible, inert material coated on one side with a continuous material comprising discontinuous regions of ECM for the manufacture of a medical device for the treatment of hernia.

Another related aspect of the invention relates to a method for treating hernia comprising the step of placing a mesh comprising a biocompatible, inert material coated on one side with a continuous material comprising discontinuous regions of ECM, in the patient covering the site of the hernia, with the coated side towards the abdominal wall.

In one aspect of the invention, the inert material is elongated and coated on both ends. This is particularly advantageous for use as slings. Here, the mesh will adhere to the anchorage points in the ends of the sling, and still allow urethral redistribution as a consequence of bladder emptying.

A related aspect of the invention relates to the use of a elongate mesh comprising a biocompatible, inert material coated in both ends, leaving a central portion un-coated, with a continuous material comprising discontinuous regions of ECM for the manufacture of a medial device for the treatment of urinary incontinence.

Another related aspect of the invention relates to a method for treating urinary incontinence comprising the step of placing an elongated mesh comprising a biocompatible, inert material coated in both ends, leaving a central portion un-coated, with a continuous material comprising discontinuous regions of ECM around the urethra such that the central portion surrounds the urethra and the ends enables anchoring.

Partial coating on one side, or to specific parts, can be obtained by dip coating, spraying techniques or brushing techniques.

In one aspect of the invention, the inert material is fully covered by the continuous material comprising discontinuous regions of ECM. This is particularly advantageous for use in implants where adherence to both side is desired. This is useful inter alia for meshes for the treatment of pelvic prolaps, reconstruction of bladder walls or Vaginal wall repair.

A related aspect of the invention relates to the use of a mesh comprising a biocompatible, inert material fully coated with a continuous material comprising discontinuous regions of ECM for the manufacture of a medial device for the treatment of pelvic prolaps.

Another related aspect of the invention relates to a method for treating pelvic prolaps comprising the step of placing a mesh comprising a biocompatible, inert material fully coated with a continuous material comprising discontinuous regions of ECM at the site of prolaps.

It is preferred that the continuous material comprising discontinuous regions of ECM is biodegradable. That is, the material disappears; is hydrolysed, is broken down, is biodegraded/bioresorbable/bioabsorbable/bioerodable, is dissolved or in other ways vanish from the biocompatible, inert material. The biodegradable region are replaced by newly synthesized host tissue thereby anchor the inert material.

It is typically preferred that the continuous material is broken down during 1 day to 10 weeks—depending on the application.

By a continuous material with discontinuous regions of ECM is understood that a first material is continuous. That is, it has a continues phase. A continuous material with discontinuous regions results in a composite material. As with other composite materials, this is an engineered material made from two or more constituent materials with significantly different physical or chemical properties and which remains separate and distinct within the finished structure.

A discrete phase of ECM material, that is a discontinuous regions of ECM, means material of ECM that is distinguished in their form and density from the ground material that they are embedded in. This can be demonstrated by histology sections as seen in example 5 or by scanning electron microscope (SEM) seen in example 6. By adding discontinuous regions of ECM, we can control the concentration of ECM.

It is preferred, that the ECM material is added to the coating before formation for the continuous material (e.g. freeze-drying). In this way, the ECM material is homogeneously distributed in the coating. That is, in the time it takes to solidify the coating (e.g. during freezing) the density of ECM material might be somewhat higher in one end of the coating than in the other. However, in the present context a homogeneous distribution allows for such density gradient through the coating provided that the density in the centre of the coating is >0. Thus, a preferred embodiment relates to a coating wherein the discontinuous regions of ECM are homogeneously distributed.

Extracellular matrix (ECM) is the non-cellular portion of animal or human tissues. The ECM is hence the complex material that surrounds cells. Consequently, it is preferred that the discontinuous regions of ECM are cell free regions. Cell free regions are obtained by the use of physical, enzymatic, and/or chemical methods. Layers of cells can be removed physically by e.g. scraping the tissue. Detergents and enzymes may be used to detach the cells from one another in the tissue. Water or other hypotonic solutions may also be used, since hypotonicity will provoke the cells in the tissue to burst and consequently facilitate the decellularization process.

Another way to obtain cell free regions is by adding the ECM powder (discontinuous regions of ECM) to the coating matrix. A cell-free product minimizes the risk any immune rejection once implanted, since components of cells may cause an immunogenic response.

In broad terms there are three major components in ECMs: fibrous elements (particularly collagen, elastin, or reticulin), link proteins (e.g. fibronectin, laminin), and space-filling molecules (usually glycosaminoglycans). ECMs are known to attract cells and to promote cellular proliferation by serving as a reservoir of growth factors and cytokines (Hodde, J. P., Record, R. D., Liang, H. A., & Badylak, S. F. 2001, “Vascular endothelial growth factor in porcine-derived extracellular matrix”, Endothelium 2001; 8.(1):11-24., vol. 8, pp. 11-24; Voytik-Harbin, S. L., Brightman, A. O., Kraine, M. R., Waisner, B., & Badylak, S. F. 1997, “Identification of extractable growth factors from small intestinal submucosa”, J. Cell Biochem., vol. 67, pp. 478-491). A coating containing particulate ECMs will be populated by cells both from the surrounding tissue as cells from the circulating blood. As the cells invade the coating new tissue is formed.

Preferred ECM materials contain bioactive ECM components derived from the tissue source of the materials. For example, they may contain Fibroblast Growth Factor-2 (basic FGF), Transforming Growth Factor-beta (TGF-beta) and vascular endothelial growth factor (VEGF). It is also preferred that ECM base materials of the invention contain additional bioactive components including, for example, one or more of collagens, glycosaminoglycans, glycoproteins and/or proteoglycans. The ECM may include the basement membrane, which is made up of mostly type IV collagen, laminins and proteoglycans. The ECM material of the invention is preferably prepared from tissue harvested from animals raised for meat production, including but not limited to, pigs, cattle and sheep. Other warm-blooded vertebrates are also useful as a source of tissue, but the greater availability of such tissues from animals used for meat production makes such tissue preferable. Pigs that are genetically engineered to be free of the galacatosyl, alpha 1,3 galactose (GAL epitope) may be used as the source of tissues for production of the ECM material. In a preferred embodiment the ECM will be of porcine origin.

The ECM material can be obtained from any animal. It could be derived from, but not limited to, intestinal tissue, bladders, liver, spleen, stomach, lymph nodes or skin. ECM derived from human cadaver skin, porcine urinary bladder submucosa (UBS), porcine urinary bladder matrix (UBM), or porcine small intestinal submucosa (SIS) are particularly preferred.

Human tissue is preferably avoided to minimize transfer of diseases. Thus, in a preferred embodiment the discontinuous regions of ECM are obtained from animal tissues. Due to species similarity, it is preferred to use ECM from warm-blooded mammal.

In a particular preferred embodiment the discontinuous regions of ECM are UBM (Urinary Bladder Matrix) particles. The UBM material comprise a unique cocktail of ECM proteins of which a few have been quantified: TGF-β 293±8 pg/g, b-FGF 3862±170 pg/g, and VEGF 475±22 pg/g (that is pg VEGF/g UBM). With an average density of 3 mg/cm², the concentration is about TGF-β: 0.9 pg/cm² in an ECM sheet, b-FGF: 11.6 pg/cm² and VEGF 1.4 pg/cm².

The concentration of the discontinuous regions of ECM is preferably higher than 15% (w/w), that is higher than 20% (w/w), such as higher than 30% (w/w). The concentration of the discontinuous regions of ECM is preferably lower than 95% (w/w), that is lower than 90% (w/w), such as lower than 80% (w/w), or lower than 70% (w/w). In a particular preferred embodiment of the invention the concentration is between 20% (w/w) and 60% (w/w), such as between 20% (w/w) and 40% (w/w).

In one aspect of the invention, the continuous material comprising discontinuous regions of ECM is made of protein containing substances such as zein, gelatine, collagen keratin, S-sulfonated keratin, fibrin, laminin, elastin or other structural proteins.

In one aspect of the invention, the continuous material comprising discontinuous regions of ECM is made of polysaccharides containing substances such as alginates, chitosan/chitin, hylaronic acid, CMC, HEC, HPC or other functionalised celluloses.

In one aspect of the invention, the continuous material comprising discontinuous regions of ECM is made of synthetic polymers containing substances such as:
a) Homo- or copolymers of: glycolide, L-lactide, DL-lactide, meso-lactide, ε-caprolactone, 1,4-dioxane-2-one, -valerolactone, β-butyrolactone, γ-butyrolactone, γ-decalactone, 1,4-dioxepane-2-one, 1,5,8,12-tetraoxacyclotetradecane-7-14-dione, 1,5-dioxepane-2-one, 6,6-dimethyl-1,4-dioxane-2-one, trimethylene carbonate.
b) Block-copolymers of mono- or difunctional polyethylene glycol and polymers of a
c) Block copolymers of mono- or difunctional polyalkylene glycol and polymers of a
d) Blends of the above mentioned polymers
e) Blends of the above mentioned polymers and PEG
   or any combinations of the above.

An MPEG-PLGA polymer can be synthesized as follows: MPEG, DL-lactide, glycolide and 4% (w/v) stannous octanoate in toluene are added to a vial in a glove box with nitrogen atmosphere. The vial is closed, heated and shaken until the contents are clear and homogeneous and then placed in an oven at 120-200° C. for 1 min-24 h. The synthesis can also be made in a solution in a suitable solvent (e.g. dioxane) to facilitate the subsequent purification. Then MPEG, DL-lactide, glycolide, 4% Stannous 2-ethylhexanoate and dioxane are added to a vial in a glove box with nitrogen atmosphere, and treated as above.

The polymer can be purified as follows: The polymer is dissolved in a suitable solvent (e.g. dioxane, tetrahydrofuran, chloroform, acetone), and precipitated with stirring in a non-solvent (e.g. water, methanol, ethanol, 1-propanol or 2-propanol) at a temperature of −40° C.-40° C. The polymer is left to settle, solvent discarded and the polymer is dried in a vacuum oven at 40° C.-120° C./overnight.

One function of the coating, at least partly covering the biocompatible, inert material is to provide a matrix promoting cell growth. One criterion to promote cell in-growth into the coating is a coating that is solid at room temperature. That is, the coating has a fixed physical structure, a bi-continuous structure. By this structure, cells are helped to migrate through the coating and form new tissue.

Another criterion to promote cell growth is a coating that has open pores, or at least a porosity that allows cell migration.

Porosity is defined as P=1−ρ(V/M)

where P is the coating porosity, ρ the density of the polymeric system used, M the weight, and V the volume of the fabricated coating.

One embodiment of the invention relates to a coating, at least partly covering a biocompatible, inert material, comprising discontinuous regions of ECM as described herein. A porosity of more than 50% enables cell growth. Thus, in a preferred embodiment the coating as described comprises a porosity of more than 50%, such as >80%, even more than 90%, or as porous at 95%.

It is preferred that the porous coating has open interconnected pores.

The thickness of the coating has to balance the ability to provide sufficient ingrowth of cells to anchor the mesh, but at the same not to be bulky and produce obstacles within the body. Thus, it is preferred that the thickness of the coating is 0.05-1 mm.

In many of these uses, it is a requirement that the mesh according to the invention is sterilized. One embodiment of the invention relates to a sterilised mesh with a coating comprising discontinuous regions of ECM. This is typically expressed as a mesh comprising a biocompatible inert material at least partly covered with a continuous material comprising discontinuous regions of ECM packaged bacterial tight, with a marking on the packaged that this product is sterilized. As illustrated in Example 4, sterilisation by e.g. radiation maintains the biological effect of ECM—dependent on coating type. Bacterial tight materials are well known to the skilled person.

### EXAMPLES

### Materials

### MPEG-PLGA Scaffold Formation

Purification of reagents: Ethyl acetate is distilled from calcium hydride under nitrogen. Dioxane is distilled from sodium/benzophenone under nitrogen. Toluene is distilled from sodium/benzophenone under nitrogen. DL-lactide and glycolide are recrystallized in dry ethylacetate in a nitrogen atmosphere and dried with vacuum. PEG/MPEG is dissolved in a suitable solvent (e.g. chloroform), precipitated in cold hexane, filtered, and dried overnight. Stannous 2-ethylhexanoate is vacuum-distilled and stored under nitrogen.

Synthesis of 2-30: 0.5 g MPEG2000, 4.15 g DL-lactide, 3.35 g glycolide and 4% (w/v) stannous octanoate in toluene are added to a vial in a glove box with nitrogen atmosphere. The vial is closed, heated and shaken until the contents are clear and homogeneous and then placed in an oven at 120-200° C. for 1 min to 48 hours, e.g. up to 6 h.

The synthesis can also be made in a solution in a suitable solvent (e.g. dioxane) to facilitate the subsequent purification. Then 0.5 g MPEG2000, 4.15 g DL-lactide, 3.35 g glycolide and 101 μL 4% (w/v) stannous octanoate and 8 g dioxane are added to a vial in a glove box with nitrogen atmosphere, and treated as above.

Purification of polymer: The polymer is dissolved in a suitable solvent (e.g. dioxane, tetrahydrofuran, chloroform, acetone), and precipitated with stirring in a non-solvent (e.g. water, methanol, ethanol, 1-propanol or 2-propanol) at a temperature of −40 to 40° C. The polymer is left to settle, solvent discarded and the polymer is dried in a vacuum oven at 40-120° C./overnight.

The polymers are analyzed with NMR-spectroscopy and GPC to confirm structure, molecular weight and purity.

### Example 1

### Construction of a Mesh with a Coated and an Uncoated Surface

25 g MPEG-PLGA (as described above) is transferred to a 100 ml measuring flask. The measuring flask is filled ¾ with 1,4-dioxane. The MPEG-PLGA is dissolved overnight at 50° C. 2.5 g of PEG400 is added to the measuring flask and the flask is afterwards filled to the 100 ml level-marker.

The MPEG-PLGA solution is transferred to a 250 ml beaker and 5 g UBM powder (e.g. ACell) is suspended in the solution using a magnetic stirrer. The UBM suspension is brushed gently on one of the major surfaces of an approximately 1.5 mm thick oxygen-plasma treated polypropylene mesh. The propylene mesh is kept at a temperature lower than 10° C. for freezing MPEG-PLGA solution and avoiding strikethrough to the other side of the polypropylene mesh. The 1.4-dioxane is removed by freeze-drying leaving a porous MPEG-PLGA coating containing ECM particles.

### Example 2

### Construction of a Fully Coated Composite Material

4 g MPEG-PLGA (as described above) is transferred to a 100 ml measuring flask. The measuring flask was filled ¾ with 1,4-dioxane. The MPEG-PLGA is dissolved overnight at 50° C. 0.4 g of PEG400 is added to the measuring flask and the flask is afterwards filled to the 100 ml level-marker. Instead of PEG400, PEG700 could be used.

The MPEG-PLGA solution is transferred to a 250 ml beaker and 2 g UBM powder (e.g. ACell) is suspended in the solution using a magnetic stirrer. 7.5 ml of the suspension is poured into a 10×10 cm aluminum mould resulting in a suspension height of 0.75 mm. A 10×10 cm polypropylene mesh with an approximate height of 1.5 mm is placed in the mould. The mould is quenched and placed in a freeze-drier overnight.

### Example 3

### In-Growth of Primary Human Fibroblasts in Synthetic Scaffolds with and without ECM Particles

Scaffolds made of biodegradable polyesters containing UBM (Acell) particles (mean diameter of approximately 150 μm) at 40% (w/w) were compared with scaffolds without the ECM particles in a test of cell morphology and 3D growth.

Metoxy-polyethylene glycol-Poly(lactide-co-glycolide) (Mn 2.000-30.000, L:G 1:1) was dissolved in 1,4-dioxane to a 1.5% solution. For the UBM containing scaffold, 0.03 g UBM was added to 3 ml polymer solution (40% w/w drymatter), high-speed-mixed and poured in 3×3 cm mould. The solution was frozen at −5° C. and lyophilized at −20° C. for 5 h and 20° C. for approx 60 h. The samples were subsequently placed in draw (hydraulic pump) in a desiccator for 5 h.

The test of growth and morphology of seeded primary fibroblasts on the surface of the two scaffolds were evaluated.

Results from day 1, 3 and 7 were graded from 1-5, with 1 corresponding to worst case and 5 being the best. In the scaffold mixed with ECM particles the distribution and growth of cells was given a grade 5 at all days and were better than the control scaffold (graded 2½ at all days).

Conclusion: The biological activity of the powdered ECM matrix retains activity after incorporation in a synthetic scaffold, and causes a considerably better growth on the scaffold when compared to scaffold alone.

### Example 4

### Effect of Sterilisation of ECM +/− Incorporation in Scaffolds on the Cell Morphology and 3D Growth of Primary Fibroblasts

Metoxy-polyethylene glycol-Poly(lactide-co-glycolide) (Mn 2.000-30.000, L:G 1:1) was dissolved in 1,4-dioxane to a 1.5% solution. For UBM containing samples, 0.045 g non-sterilized UBM was added to 10 ml polymer solution (23% w/w drymatter), high-speed-mixed and poured in 7×7 cm mould. The solution was frozen at −5° C. and lyophilized at −20° C. for 5 h and 20° C. for approx 18 h. The samples were subsequently placed in draw (hydraulic pump) in a desiccator for 15 h.

The samples with and without UBM were beta radiated by 0, 1×25 kGy and 2×25 kGy. Another sample was prepared in the same way, but a pre-sterilized UBM (2×25 kGy beta radiation) was used (0.045 g/5 ml solution) and the sample was not sterilized after preparation.

Gelatin from porcine skin, type A, bloom 175 (Sigma) was dissolved in milli-Q water and t-BuOH (95:5) to a 1% solution. 0.015 g non-sterilized UBM was added to 5 ml solution (23% w/w drymatter) while stirring and poured into the mould (D=5 cm). The mould with the solution was placed in +5° C. for 2 h, then frozen at −20° C. and lyophilized at −20° C. for 5 h and at 20° C. for 20 h. The samples were subsequently cross-linked in vacuum oven at 120° C. for 15 h. The samples with and without UBM were beta radiated by 0 and 1×25 kGy and 2×25 kGy. Another sample was prepared in the same way without UBM. The samples were sterilized after preparation at 0, 1×25 kGy and 2×25 kGy.

Gelatin from porcine skin, type A, bloom 175 (Sigma) was dissolved in milli-Q water and t-BuOH (95:5) to a 1% solution. 0.015 g pre-sterilized UBM (1×25 kGy) was added to 5 ml solution (23% w/w drymatter) while stirring and poured into the mould (D=5 cm). The mould with the solution was placed in +5° C. for 1 h, then frozen at −20° C. and lyophilized at −20° C. for 5 h and at 20° C. for 50 h. The samples were subsequently cross-linked in vacuum oven at 130° C. for 15 h.

The cell morphology and 3D growth study showed that an increasing radiation of UBM sheets reduced the number of cells on the UBM sheets but with no effect on the morphology of the cells. In the gelatine scaffold and gelatine with 30% (w/w) UBM a decreasing number of cells and a change in morphology from typical fibroblastic cells to a more rounded one was seen with the largest effect seen in the gelatine scaffold. Sterilisation of UBM particles before incorporation in gelatine scaffolds gives a better cell morphology and 3D growth compared to incorporation of UBM particles before sterilisation of the scaffold. In the MPEG-PLGA an increasing radiation resulted in an increased number of cells with fibroblastic morphology due to increased moistening of the scaffold. Radiation of scaffolds of MPEG-PLGA containing 30% (w/w) UBM resulted in an even higher number of cells and a more 3D morphology of the fibroblasts also compared with scaffold where the UBM particles were radiated before incorporation into the scaffold.

This study showed that the highest biological activity was achieved in the non-radiated gelatine scaffold and that radiation decreased the activity. On the contrary the highest biological activity was found when the UBM particles were incorporated in the MPEG-PLGA scaffold, and subsequently sterilized. It is believed that radiation decreases the biological activity of UBM. Radiation can affect the scaffold material in a negative or positive way depending on the material in relation to biological activity. There are indications showing that the scaffold material (e.g. MPEG-PLGA) can have a protective effect of the UBM during sterilization.

### Example 5

### Discrete Particles of ECM in MPEG-PLGA

Scaffolds of MPEG-PLGA containing 41% (w/w) of UBM particles were seeded with primary fibroblasts on the surface of the scaffolds with a density of 2.5×10⁴ cells/cm² in a small volume of growth medium (10% FCS in DMEM containing antibiotics (penicillin, streptomycin and Amphotericin B). The scaffolds were incubated at 37° C. at 5% CO₂ before additional growth medium was added. After 7 days the scaffolds were placed in Lillys fixative for 3 days before embedding in paraffin, sectioning into 8 μm slices and staining by Meyer's haematoxylin erosion (HE). Digital images (4× and 20× magnifications) were collected using a BX-60 Olympus microscope fitted with an Evolution MP cooled colour camera (Media Cybernetics) and digital image were taken using Image Pro Plus 5.1 software.

Digital images of the distribution of ECM particles in the MPEG-PLGA scaffold showed discrete UBM particles stained red by HE and distinguish from the scaffold material. Fibroblasts growing in the scaffold were stained blue (FIG. 1).

### Example 6

### Discrete Particles of UBM in MPEG-PLGA Shown by SEM

Scaffolds were prepared as described in Example 1.

The SEM pictures are showing MPEG-PLGA scaffolds with (FIG. 3) and without (FIG. 2) UBM particles. The pictures are taken at the top surface of the scaffold at a magnitude of 250. The SEM pictures were taken at the Danish technological institute (2005-160)

### Example 7

### Three Dimensional Endothelial Growth and Differentiation in Scaffolds Holding ECM Particles

Metoxy-polyethylene glycol-Poly(lactide-co-glycolide) (Mn 2.000-30.000, L:G 1:1) was dissolved in 1,4-dioxane to a 1.5% solution. For UBM containing samples, 0.045 g non-sterilized UBM was added to 10 ml polymer solution (23% w/w drymatter), high-speed-mixed and poured in 7×7 cm mould. The solution was frozen at −5° C. and lyophilized at −20° C. for 5 h and 20° C. for approx 16 h. The samples were subsequently placed in draw (hydraulic pump) in a desiccator for 15 h.

Primary human endothelial cells from umbilical cord were co-cultured with primary human dermal fibroblasts on the surface of MPEG-PLGA scaffolds and scaffold containing 23% (w/w) UBM. The constructs were cultured submerged in defined endothelial growth medium for 6-10 days after which they are airlifted and cultured for another 9 days. On the final day of culture constructs were fixed with 4% formalin buffer, bisected and paraffin embedded.

By immunohistochemical peroxidase staining of CD31/PECAM (platelet endothelial cell adhesion molecule) endothelial cells were visualized on 5 μm sections. Identifying fibroblasts, parallel sections were stained with PECAM peroxidase combined with a haematoxylin counterstain. As endothelial growth and differentiation is influenced by fibroblast performance, all scaffold materials were tested with 2 different fibroblast populations but were not giving rise to different results.

All MPEG-PLGA scaffolds support fibroblast and endothelial growth. Fibroblasts were found throughout the entire volume of all MPEG-PLGA scaffolds. UBM particles were homogenously distributed and scaffolds remain intact during culture. Culturing endothelial cells and fibroblasts on MPEG-PLGA scaffolds however brings endothelial surface growth only—endothelial cells proliferate within a matrix produced by the neighboring fibroblasts on top of the scaffold. Adding UBM particles promote fibroblast and endothelial growth in the deeper layers of the scaffolds and endothelial cells adopt capillary-like morphology. Endothelial cells are guided along the surface of UBM particles rather than migrating into them. Therefore we find that including UBM particles in scaffolds lead to a very distinct improvement in endothelial growth and differentiation. The different fibroblast populations were not giving rise to different results.

MPEG-PLGA scaffolds (FIG. 4) and 23% (w/w) UBM in MPEG-PLGA (FIG. 5) show growth of endothelial cells in the surface of the MPEG-PLGA scaffold where the growth is into the depth holding UBM particles (endothelium is stained red (shown black)—fibroblasts are not visible).

Capillary-like morphology of endothelial cells were seen in the deeper layer of MPEG-PLGA scaffold holding 23% (w/w) UBM (FIG. 6). These structures were not seen in the MPEG-PLGA scaffold.

### Example 8

### Discrete Particles of ECM in a Bicomponent Biocompatible Inert Fibre

20 g ECM is compounded into a 180 g Tecoflex® (EG-80A) from Noveon using a Dr. Collin extruder operating at 150° C.-180° C. The compound is granulated using a inline peletiser giving 200 g urethane pellets containing approximately 10% ECM particles.

The compounded Tecoflex® granulates is feted to a modified FET laboratory fibre coextruder. An 0.2 mm diameter oxygen-plasma treated PP fibre was feted to the centre and the compounded Tecoflex® is co-extruded on to the PP fibre resulting in a 0.3 mm diameter monofilament. The bicomponent monofilament may be stretched afterwards in order to reduce the final diameter of the monofilament.

This bicomponent monofilament contains accessible ECM particles at the surface of the Tecoflex®.

### FIGURES

FIG. 1: Digital images of the distribution of ECM particles in the MPEG-PLGA scaffold.

FIG. 2: SEM picture of MPEG-PLGA scaffold (Magnification 250×).

FIG. 3: SEM picture of MPEG-PLGA containing 40% ECM particles (Magnification 250×).

FIG. 4: Digital image of endothelial growth in MPEG-PLGA scaffold.

FIG. 5: Digital image of endothelial growth in MPEG-PLGA containing 23% ECM particles.

FIG. 6: Digital image of endothelial growth in MPEG-PLGA containing 23% ECM particles showing a magnification of capillary-like morphology in the deeper layers of the scaffold.

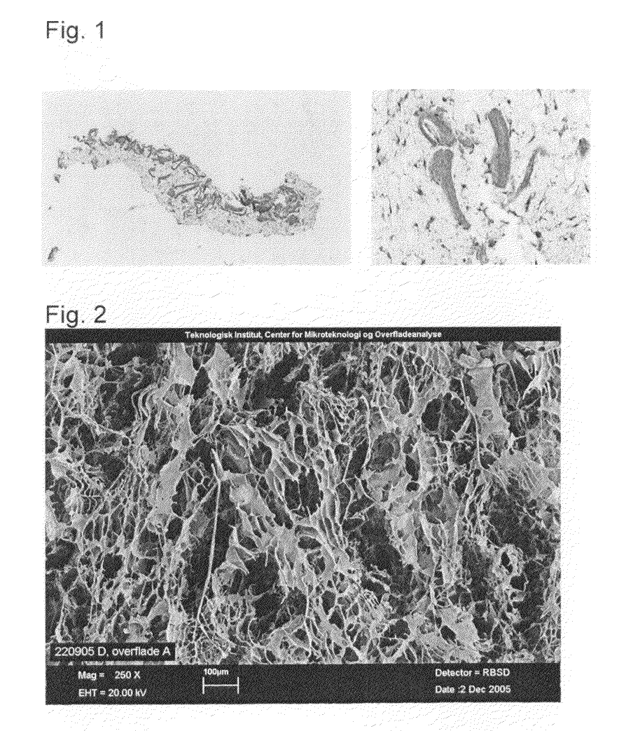

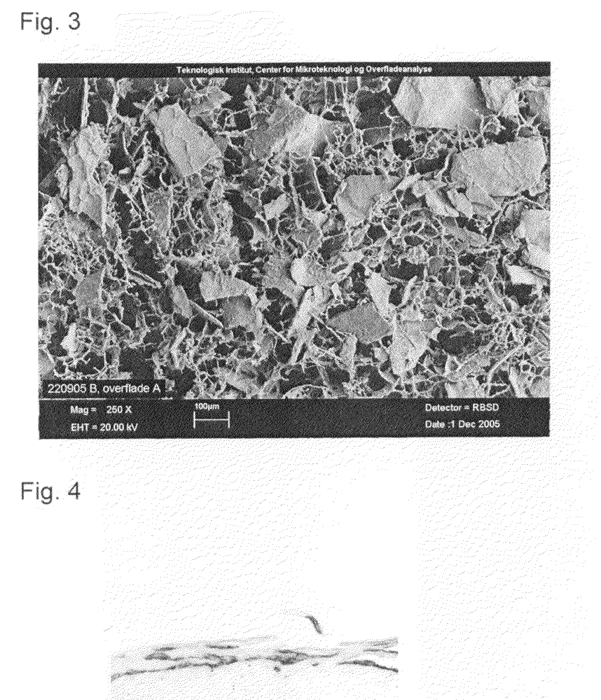

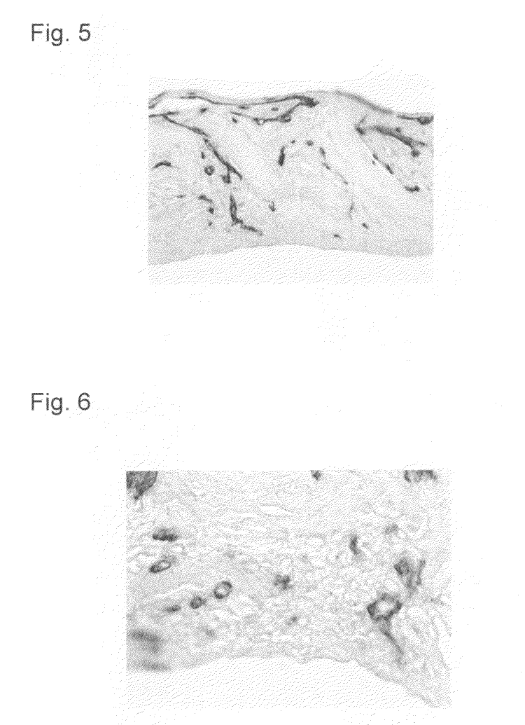

1. A mesh comprising a biocompatible inert material at least partly covered with a continuous material comprising discontinuous regions of ECM.

2. The mesh according to claim 1, wherein the biocompatible inert material is selected from the group consisting of PP, PE, polymers obtained by metallocene catalyzation, silicone, Teflon (fluoro carbons) and polyurethanes.

3. The mesh according to claim 1, wherein the biocompatible inert material is poly propylene.

4. The mesh according to claim 1, wherein the inert material is coated on one side with the temporary, continuous material comprising discontinuous regions of ECM.

5. The mesh according to claim 1, wherein the inert material is fully covered by the continuous material comprising discontinuous regions of ECM.

6. The mesh according to claim 1, wherein the continuous material is biodegradable.

7. The mesh according to claim 1, wherein the discontinuous regions of ECM are homogeneously distributed.

8. The mesh according to claim 1, wherein the continuous material has open interconnected pores.

9. The mesh according to claim 1, wherein the continuous material has a thickness of 0.05-1 mm.

10. The mesh according to claim 1, wherein the mesh is packaged bacterial tight, with a marking on the packaged that this product is sterilized.

11. A mesh comprising a biocompatible inert material with discontinuous regions of ECM particles at the surface.

12. A method for treating hernia comprising the step of placing a mesh comprising a biocompatible, inert material coated on one side with a continuous material comprising discontinuous regions of ECM, in the patient covering the site of the hernia, with the coated side towards the abdominal wall.

13. A method for treating urinary incontinence comprising the step of placing an elongated mesh comprising a biocompatible, inert material coated in both ends, leaving a central portion un-coated, with a continuous material comprising discontinuous regions of ECM around the urethra such that the central portion surrounds the urethra and the ends enables anchoring.

14. A method for treating pelvic prolaps comprising the step of placing a mesh comprising a biocompatible, inert material fully coated with a continuous material comprising discontinuous regions of ECM at the site of prolaps.
